(19) United States
(12) Patent Application Publication
(10) Pub. No.: US 2024/0130998 A1
(43) Pub. Date: Apr. 25, 2024

(54) ANALGESIA FROM ORAL PROCAINE A FORGOTTEN VOLTAGE GATED SODIUM CHANNEL BLOCKER

(71) Applicant: Joel Steven Goldberg, Hillsborough, NC (US)

(72) Inventor: Joel Steven Goldberg, Hillsborough, NC (US)

(21) Appl. No.: 18/400,025

(22) Filed: Dec. 29, 2023

(51) Int. Cl. A61K 31/245; A61K 9/00; A61K 31/5517; A61P 29/00

(52) U.S. Cl. CPC A61K 31/245; A61K 9/0053; A61K 31/5517; A61P 29/00

(57) ABSTRACT

This invention is a safe and effective method to treat chronic pain with the oral administration of procaine as a non-selective voltage sodium channel (VGSC) blocker. The dose of oral procaine has been estimated based upon the pharmacokinetics required to produce action potential inhibition in neurons of dorsal root ganglia. Intraneural ion trapping effects and low CSF pseudocholinesterase will increase the concentration of charged procaine within Naᵥ 1.8 channels. The co-administration of benzodiazepines will increase the therapeutic ratio of procaine mitigating central nervous system toxicity. A dibucaine number is required prior to initiation of therapy. Side effects have hindered development of selective VGSC blockers. Modification of oral procaine administration with over 100 clinical practice years of known side effects may meet this unmet need.

### CROSS-REFERENCES TO RELATED APPLICATIONS

This application claims the benefit of U.S. application Ser. Nos. 14/167,028, 15/463,205, 16/876,647, and 17/698,053.

### FEDERALLY FUNDED RESEARCH

Not applicable

### BACKGROUND OF THE INVENTION

### Definitions

1. Procaine refers to any salt of procaine including procaine hydrochloride unless otherwise specified.

2. Plasma esterase refers to pseudocholinesterase which is found in blood and cerebral spinal fluid (CSF).

Chronic Pain is an Excitatory Neural Process

The evidence is quite compelling that chronic pain is a neural excitatory process which causes an immense amount of human suffering. This proposition is supported by neural conduction block or lesioning of excitatory neural components, either centrally or peripherally, that diminishes or extinguishes chronic pain. Electrophysiology of nociceptors reveals increased firing in chronic pain states, and stimulation of inhibitory nerves and neurons diminishes pain perception. Pharmacologic interventions, which are the focus of this invention, are directly or indirectly inhibitory on the net propagation of nociception action potentials that reach somatosensory regions of the brain.

Transduction-Neural Mechanisms of Chronic Pain

Stimuli that produce pain do so by activation of nerves or neurons which is called transduction. The process can be interrupted directly by four major pharmacologic pathways:
1. G protein coupled receptor (GPCR) interactions which are the basic mechanism for opioids, non-steroidal anti-inflammatory (NSAIDS), acetaminophen, antidepressants, and cannabinoids;
2. Interruption of energy sources by antiglycolytic agents;
3. Interruption of action potentials by ligands of gated channels such as ketamine, methadone, or dextromethorphan,
4. and interruption of action potentials by block of voltage gated channels mainly of sodium by anticonvulsants, local anesthetics, and other ligands.

Opioids

Opioids are effective analgesics for chronic pain; however, their use is associated with a myriad of side effects, including abuse, addiction, respiratory depression with death, constipation, immune deficiency, and pruritus. Opioids primary mechanism of action as a ligand to GPCR is inhibition of intracellular adenylyl cyclase and formation of cyclic adenosine monophosphate (cAMP). The worldwide opioid epidemic that occurred as a result of long term opioid treatment of chronic pain was a miscalculation of medical therapy, and it was assumed the public and physicians could control the side effects of addiction and abuse. The political outcry from opioid related deaths led to prosecution of some parties who in many cases were peripherally involved with the administration of opioids but had assets that could be acquired by successful litigation. Very few prescribers had intentions to hurt their patients who suffered from intractable pain by prescribing opioids. The dramatic number of opioid overdoses was not predicted at the time to occur with chronic opioid administration. One utility of this invention is a method to spare opioid prescribing with administration of oral procaine. Ironically, Einhorn in 1905 developed procaine to spare prescription of addictive cocaine.

NSAIDS

NSAIDS produce analgesia through inhibition of the two isoforms of cyclooxygenase which changes the ratio of prostanoids that are GPCR ligands.

Antidepressants

Antidepressants, mostly tricyclic antidepressants and selective norepinephrine serotonin reuptake inhibitors, produce analgesia as GPCR ligands, although some antidepressants such as amitriptyline are voltage gated channel blockers, presumably from intraneural quaternary amine block of sodium channels.

Acetaminophen

Although the mechanism of action of acetaminophen is not fully elucidated, acetaminophen and its metabolites primarily produce analgesia as ligands of GPCR.

Cannabinoids

Cannabinoids produce analgesia as GPCR ligands associated with diverse physiologic effects.

Summary of GPCR Ligand Analgesics

GPCR provide a mechanism for a pharmaceutical to affect cell function by providing transduction across cell membranes, which is one of the most common mechanisms of drug action. However, once the ligand signal is transduced, the complex cascade of intracellular reactions that occurs has only been partially elucidated. Therefore, it is not surprising that pharmaceuticals, whose mechanism of action is via GPCR produce, a myriad of side effects.

Antiglycolytic Agents

D-Lactic Acid Dimer produces topical analgesia through a mechanism of sequestration of L-Lactate.[1, 2] The combination of D-Lactic Acid Dimer and 2-Deoxy-D-Glucose has been predicated to produce analgesia through an antiglycolytic mechanism to decrease available adenosine triphosphate (ATP) for generation of action potentials, but this analgesic combination has not been proven.[3, 4]

Ligand Gated Activated Channels

The N-methyl-D-aspartate (NMDA) glutamate receptor is the primary excitatory ligand activated channel in the human central nervous system (CNS) associated with chronic pain. Antagonists of this receptor include ketamine, methadone, and dextromethorphan. Conversely, stimulation of inhibitory ligand active channel receptors of glycine and gamma amino butyric acid (GABA) produces analgesia, although clinically useful compounds are not commonly prescribed.

Voltage Gated Channel Blockers.

There exist multiple voltage gated channel blockers including sodium, potassium, calcium, and chloride. Voltage gated sodium channels (VGSC), which are most intimately associated with chronic pain, exist within the membranes of peripheral nerves and within the membranes of neurons. The VGSC have been structurally characterized, and computer design has been extensively utilized to develop lead pharmacophores. The VGSC consist of four homologous transmembrane domains, and each domain has 6 segments. The physio anatomy of the VGSC is sensing units (segments S1-S4, S5-6, and a central channel or pore that permits sodium to enter the cell). These sites have been further characterized as the central pore cavity site, the upper selectivity filter binding site, the resting voltage sensory domain site two (VSDII), and the active voltage sensory domain site five (VSDV).[5] VGSC blockers produce analgesia via block of sodium influx into nerves and neurons which interrupt their generation of action potentials. This block can be direct at the channel or indirect via the voltage sensors. The sites most often associated with chronic pain are commonly named Na v 1.7, Na v 1.8, and Na v 1.9 channels. Variants of these sodium channels have been found in 17% of patients with small fiber neuropathy, and Na v 1.8 channel is found in 90% of dorsal root ganglia nociceptors, so it is an active target for pharmacophores. Synthesis of Naᵥ channel blockers at the central pore cavity include Vixotrigine (Convergence/Biogen), VX-548 (Vertex), PF-01247324 (Pfizer), PF-04531083 (Pfizer) and A-803467 (Abbott/lcagen), none of which is an FDA approved medication at the present time.[5] The distribution and activity of VGSC varies considerably among species which has impeded the development of selective blockers.
Naᵥ 1.7 vs Naᵥ 1.8 vs Naᵥ 1.9 Channels[6]

Naᵥ 1.7 channels are tetrodotoxin sensitive. They can be blocked from outside the neural membrane, unlike the intraneural block from procaine, and this channel is mostly suspected to amplify the action potential. The Naᵥ 1.9 channel is tetrodotoxin resistant with sodium blocking that is intraneural; however, the Naᵥ 1.9 channel is believed to contribute minimally to generation of the action potential. The Naᵥ 1.8 channel is tetrodotoxin resistant with a mechanism of action at the intraneural inner pore, and it is a major contributor to the generation of action potentials.

Anatomy of the Human Dorsal Root Ganglia[7]

The dorsal root ganglia lie in an area near the intervertebral foramina and are surrounded by dura and cerebral spinal fluid, although there is blood supply through fenestrated capillaries that is not regulated by the blood brain barrier (BBB). Within the ganglia, there exists a number of different cell types including T- and B-lymphocytes, macrophages, and satellite glia, but those most pertinent to this invention are neurons with VGSC that propagate action potentials encoding information of chronic pain. The human intraneural pH within dorsal root ganglia neurons is not known, however the pH of the cervical ganglia of a rodent has been measured at 6.51.[8] In general, intraneural pH decreases significantly with generation of action potentials.

This invention focuses on block of the central channel in VGSC, which is the predominant action of local anesthetics to include procaine, 2-chloroprocaine, and lidocaine. Of note and common in pharmacology, the literature describes a plethora of biological actions for local anesthetics, and in particular procaine, to include vitalization, anti-cancerous, anti-rheumatic, anti-inflammatory, anti-arrhythmic, broncho-spasmolytic, sympathico-lytic, and vascular dilatation.[9] Procaine has also been shown to bind or antagonize the function of NMDA receptors as well as nicotinic acetylcholine receptors and the serotonin receptor-ion channel complex. These actions are secondary to the primary action of procaine, which is inhibition of action potential generation via VGSC block.

Other pharmaceuticals that internally block the VGSC after being transported across the cell membrane as lipophilic agents include amitriptyline, carbamazepine, and dexmetatomidine, each possessing a quaternary intracytoplasmic amine.

Over the last few decades, millions of US dollars have been spent for drug development of selective VGSC blockers which has been hampered mostly by unacceptable side effects. The utility of this invention takes a different approach to VGSC blocker development with the modification of administration of oral procaine or its equivalent, which are known low toxic drugs. Such an approach uses the extensive clinical knowledge of procaine acquired over more than one hundred years as compared to the unknown actions and side effects of new design selective VGSC blocking drugs that have problems with bioavailability, distribution, metabolism, and adverse effects. If administrated according to the methods of this invention, oral procaine or its equivalent as a VGSC blocker is a treatment for chronic pain with clinically acceptable side effects.

Prior Art-Procaine Analgesia

There is a myriad of studies that describes a multitude of pharmaceutical properties of procaine. This invention limits a review of the prior art to procaine analgesia with a focus on oral administration of procaine or its equivalent for the treatment of chronic pain in human subjects.

Intravenous Procaine Analgesia

Intravenous (IV) procaine analgesia has been known for decades, and IV procaine with nitrous oxide is a successful general anesthetic. Seasoned pain professionals anecdotally acknowledge that IV administration of local anesthetics, to include procaine, 2-chloroprocaine, and lidocaine, produces analgesia in most patients suffering from intractable chronic pain prior to side effects of convulsions or cardiac arrhythmias.[10] However, there are many reports that IV procaine has minimal analgesic efficacy, and these studies teach away from this invention.

Keats, A. S. et al. reported inadequate analgesia with IV procaine or IV procaine with ascorbic acid in 53 patients suffering from acute post-operative pain with an approximate dose of 370 mg. They concluded, “The analgesia produced by intravenous procaine was accompanied by frequent unpleasant and sometimes serious side-actions. Unless procaine, administered by this route, can be demonstrated to produce significant beneficial effects on specific disease-processes, it has no place in the treatment of pain.”[11] In Germany, IV procaine had been administered for the treatment of acute pancreatic pain until Kahl, S. reported poor analgesia with an infusion at a dose of 2000 mg over 24 hours.[12] However, Zeluff, R. J. M. reported dramatic relief of acute pain with IV procaine in 3 patients with a dose of 1000 mg in traumatic military injuries.[13] Shanbrom, E. reported chronic pain relief in 13/16 patients with postherpetic neuralgia treated with 500 mg to 1000 mg IV procaine. [14] Betcher, A. M. reported adequate relief of pain and discomfort from IV procaine in 92 patients with various orthopedic conditions.[15]

Procaine pharmacokinetics and pharmacodynamics were discovered subsequent to many of these reports, and predicted calculations of plasma procaine concentrations in these studies were likely sub therapeutic, which explains the conclusions. For example, with the known distribution and elimination half-lives of ˜2.50 and 7.70 minutes respectively, and average volume of distribution of 0.57 L/kg after steady state IV procaine administration, it could require an initial bolus loading dose of 400 mg and bolus dosing of 200 mg approximately every 3 minutes to achieve what was considered a therapeutic blood level of 10 μg/ml.[16] It is clear that some of IV procaine analgesic studies did not achieve sufficient levels to be an effective analgesic, which questions the conclusions.

Oral Procaine Analgesia

Reports of oral procaine for treatment of chronic pain are not extensive and contradictory.

TABLE 1
Oral procaine analgesia

| Investigator | Dose | | Efficacy |
|---|---|---|---|
| Klein, R. et al. 1953[17] | 450 | mg TID | 38/50 |
| Kupperman et al. 1957[18] | 1000 | mg TID | 0/50 |
| Koch, R. 1989[19] | 200 | mg BID | 29/30 |
| Balfour et al. 1951[20] | 80 | mg | 8 |
| Beinhauer et al. 1954[21] | 3000 | mg/day | 9/14 |
| Traut, E.F. 1952[22] | 3000 | mg/day | 5/32 |

Klein reported excellent analgesia from oral procaine in 38/50 patients suffering chronic pain from arthritis, while Kupperman reported no analgesia from oral procaine in 50 patients with arthritis.[17] Kupperman administered the procaine with meals while Klein did not specify. Administration of procaine with meals could have affected oral bioavailability if the food contained esterases or if the pH of the stomach became increasingly acidic predisposing to ester hydrolysis of the procaine. Kupperman reported peak plasma procaine levels of 0.2 mg percent (2 mg/ml) to 3.1 mg percent (31 mg/ml), which in light of present methods, must be erroneous. Koch administered a complex of procaine HCl, nicotinic acid, folic acid, biotin, ascorbic acid, citric acid, potassium, and magnesium described as a double salted procaine with excellent pain relief in 29/30 with chronic pain from rheumatoid arthritis.[19] Balfour administered oral procaine with Metamucil in 8 patients for successful treatment of chronic dysphagia.[20] Beinhauer treated patients with chronic pruritus with oral procaine and found that 9/14 patients with herpes zoster or burning tongue syndrome had excellent relief of symptoms.[21] Traut reported chronic pain relief in 5/32 patients with arthritis who were prescribed a total of 3000 mg/day of oral procaine in divided doses after meals, but claimed that after 2000 mg of oral procaine, none could be detected in the blood.[22] Unlike the methods in this invention, patients were not prescreened for plasma esterase activity nor pretreated with an anticonvulsant medication to prevent convulsions.

Except for references of Kupperman and Traut, there are no references that describe procaine blood levels from oral procaine administration, but there is enough evidence to confirm that the drug can be absorbed in the digestive system and attain blood levels. Aside from the oral analgesic studies, there are numerous studies of therapeutic oral procaine for pruritus, pyloric spasm, and asthma.[23-25]

Dibucaine Number

The dibucaine number measures the activity of pseudocholinestrase in the blood. Pseudocholinesterase hydrolyzes procaine. Dibucaine is a local anesthetic that inhibits the activity of pseudocholinesterase. The dibucaine number will determine if the patient is homozygous dominant for plasma esterase (70-80), heterozygous dominant for plasma esterase (30-70), or homozygous for non-dominant plasma esterase (<30). The dominant gene is the wild type with normal pseudocholinesterase activity and expression.

Oral Procaine Pharmacokinetics

Procaine is bioavailable from the digestive system in spite of its labile ester bond, and procaine is hydrolyzed by plasma esterases in the blood and CSF. Plasma esterase levels greatly affect the pharmacokinetics of procaine. In the blood, procaine can be hydrolyzed to para-aminobenzoic acid (PABA) and diethylaminoethanol (DEAE), transported into the interstitial space to nerves and tissues of cardiac conduction, or cross the BBB where it can be either hydrolyzed or transported into the cytoplasm of neurons. It is the transported procaine into neurons that is most relevant to this invention. Prior to chronic administration of oral procaine for the treatment of chronic pain, assessment with a dibucaine number of the patient's pseudocholinesterase activity is required, and this measurement will influence the oral procaine dose. Ester hydrolysis of procaine varies greatly among mammalian species; therefore, animal models are of limited value.

Oral Procaine Pharmacodynamics

Procaine is a non-selective VGSC blocker that inhibits the generation of action potentials via internal block of sodium influx. Like most local anesthetics, procaine is transported across the cell membrane of nerves or neurons because of its lipid characteristics, and the protonated form blocks the entry of sodium into the nerve or neuron, thus preventing propagation of action potentials. Unlike tetrodotoxin, procaine blocks the entry of sodium from within the cytoplasm, not exterior to the cytoplasm. The mechanism of action of procaine within neurons has been substantiated by multiple studies and is accepted as valid. The systemic administration of procaine will not provide an intraneural procaine concentration within peripheral nerves to provide analgesia.[26] The mechanism of analgesia from systemic procaine is within the CNS to include the dorsal root ganglia.[27]

Cardiovascular Toxicity of Procaine

Procaine is one of the least cardio toxic of all the commonly used local anesthetics. In almost all cases of toxicity, the CNS toxicity precedes any cardiotoxicity. Rarely In selected cases with oral procaine therapy, the patient may need to be pretreated with a beta agonist to decrease the incidence of depression from procaine beta adrenergic effects on pacemaker cells.

Central Nervous System Toxicity of Procaine

The estimated CNS toxicity is the major side effect that limits procaine dosage. It is predicated that pretreatment or concurrent administration with a benzodiazepine will significantly increase the therapeutic ratio and permit higher levels of procaine to reach the CNS. This treatment will make possible a higher safe dose of orally administered procaine which will increase the block at dorsal root ganglia VGSC and, in particular, Na v 1.8 channels. Although such pretreatment may seem complex, the advantages far outweigh the risks. Compared to the daunting task of developing a new selective VGSC blocker with so many variables, such as absorption, distribution, metabolism, elimination, and unpredictable side effects, the extensive knowledge of over one hundred years of procaine pharmacology as a VGSC blocker clearly outweighs the cost and uncertainty of a new selective VGSC blocker.

Oral Administration of 2-Chloroprocaine

Oral administration of 2-chloroprocaine is not described in the literature. The anesthetic 2-chloroprocaine is hydrolyzed by plasma esterase at a 4× greater rate than procaine, and it is considered the least cardiac and cerebral toxic local anesthetic in medical practice.[28] One embodiment of this invention is the oral administration of 2-chloroprocaine.

Oral Administration of Lidocaine

There are numerous reports of the analgesic effects of intravenous lidocaine for the treatment of chronic pain.[10] Its primary mechanism of action is purported to be the same as procaine, that is blocking of the central pore of sodium channels. Unlike procaine, lidocaine is an amide local anesthetic, and its CNS and cardiac toxicity are significantly greater than procaine. In the liver, lidocaine is dealkylated by oxidizing enzymes to the pharmacologically active metabolite, monoethylglycinexylidide (MEGX), and then metabolized into N-ethylglycine (NEG) and glycinexylidide (GX). MEGX is 80% as potent as the parent drug, while GX is nearly ineffective. Achieving systemic lidocaine therapeutic levels with an active metabolite to block VGSC even with pretreatment of a benzodiazepine might be risky, and its oral use is not an embodiment of this invention.

### BRIEF DESCRIPTION OF THE SEVERAL VIEWS OF THE DRAWING

Not applicable

### BRIEF SUMMARY OF THE INVENTION

This invention is a safe and effective method to treat chronic pain with an oral non-selective VGSC blocker of procaine salts or its equivalents. It is unlikely that systemic administration of procaine or its equivalents produces analgesia from blocking of VGSC within afferent peripheral nerves, and the mechanism of action is blocking of VGSC within the dorsal root ganglia and, in particular, Na v 1.8 channels.[26, 27] Contrary to some prior art, not obvious to one skilled in the art, and despite procaine's short distribution and elimination half-lives, oral procaine administration can achieve, through intraneural ion trapping effects and the relative deficiency of CSF pseudocholinesterase, a therapeutic analgesic concentration within dorsal root ganglia. Many prior studies attesting to poor analgesia from IV or oral procaine were incorrect, since the dose of procaine administered were sub therapeutic, according to more recent pharmacokinetic data, or there may have been interference with oral bioavailability. Concurrent or pretreatment with a benzodiazepine is predicted to increase the therapeutic ratio of procaine mitigating central nervous system toxicity. Procaine has one of the lowest cardiac toxicities of local anesthetics. A dibucaine number to assess plasma pseudocholinesterase activity, EKG, and hypersensitivity history or testing is required prior to initiation of oral procaine therapy. For decades, pharmaceutical development of selective VGSC blockers for treatment of chronic pain has failed to bring to clinical practice a suitable drug, largely because of side effects. It seems more practical to modify administration of oral procaine where the side effect profile is well known, and procaine has been administered in clinical practice for more than one hundred years. Clinical trials are suggested.

### DETAILED DESCRIPTION OF THE INVENTION

As described in this invention, the method of oral administration of procaine for the treatment of chronic pain is not obvious. Much of the prior art teaches away from this invention, and it is only through creative conception of the prior art that one might conceive of the essence of the method. The utility of the invention is obviously an unmet need for a safe and effective oral analgesic which has eluded pharmaceutical development for decades.

To one skilled in the art, it would seem obvious that oral procaine could not be a systemic analgesic, since the central compartment distribution half-life of ˜2.5 minutes and central compartment elimination half-life of ˜7.7 minutes would preclude its use as an effective oral drug.[16] However, as one follows the pharmacokinetics and pharmacodynamics of procaine within various tissue compartments each with differing pH, ion trapping effects predict that intraneural therapeutic levels are obtainable. Procaine has a pKₐ of 8.9. The pKₐ of the quaternary amine of DEAE is ˜10.1, and the pKa of the protonated amine of PABA is pKa˜4.6, predicting a pKₐ value less than 8.9. A dicationic species of procaine has never been shown to exist. As the molecule transits from the blood to neural cytoplasm, procaine becomes more charged and less lipophilic within the cytoplasm of the neuron, and the concentration of the active charge species increases. The charged active species is essentially trapped in the cytoplasm, only being able to exit after hydrolysis to PABA and DEAE or through distribution. (Table 2) Human intraneural esterases may exist, but human intraneural pseudocholinesterase is not known.[29]
B=uncharged procaine BH⁺=charged procaine
BH⁺↔B+H⁺
pH=pKa+log B/BH⁺

The intraneural pH of human dorsal root ganglia is not known, but it is generally accepted that intraneural pH decreases with neural activity. The pH of cervical ganglia of a rodent is reported as 7.30-6.51, and the typical resting or “steady-state” pH within a hippocampal neuron is reported as ˜7.03-7.46,[30] Therefore, [BH+]/[B] will increase from blood to intraneural cytoplasm possibly by factor of at least 7.5.

TABLE 2
Ion trapping effects of charged and uncharged procaine

| Site | pH | [BH⁺]/[B] |
|---|---|---|
| Blood | 7.4 | 32 |
| Interstital | 7.4 | 32 |
| CSF | 7.32 | 38 |
| Ganglia or Intraneural[8] | 7.30-6.51 | 40-245 |

Furthermore, as procaine enters the CSF with pseudocholinesterase activity of 1/20-1/100th of the plasma, ester hydrolysis of procaine is significantly diminished, increasing both the CSF [B] and [BH⁺] relative to blood, and the kinetics of this hydrolysis is linear from 0.0 to 0.2 units of pseudocholinesterase as defined by the hydrolysis of naphthyl acetate. [31]. The intraneural activity of pseudocholinesterase is not known, but ion trapping effects and diminished CSF pseudocholinesterase activity change the [BH⁺]/[B] from blood to intraneural predicting an intraneural [BH⁺] as large as 750 times blood [BH⁺] without considering Michaelis-Menten kinetics:

[BH⁺][B]ᵢₙₜᵣₐₙₑᵤₐₗ=[BH⁺]/[B]_{blood}*7.5*100

Neurons, and in particular those within the dorsal root ganglia with an abundance of Naᵥ 1.8 channels, are particularly sensitive to local anesthetic block at a minimum concentration of 10 μM.[32] Experimental block of dorsal root ganglia neurons that occurs at a minimum concentration of 10 μM allows one to extrapolate the procaine blood level required to produce the CSF level needed for inhibition of action potential propagation.
Molecular weight of procaine=236.31 g/mol
10 μM=2.36 μg/ml

In a 70 kg human, with an average central compartment procaine blood volume of distribution of 0.57 L/kg procaine or 40.0. L, an initial blood level of 2.36 μg/ml is obtained with a 95 mg bolus IV procaine administration.[16] This concentration would rapidly distribute to the CSF with a volume of 150 ml, distribute to other compartments, and undergo elimination. Assuming a mere 10% oral procaine bioavailability, this would require an initial approximate oral procaine dose of 1000-2000 mg for a therapeutic CSF level. Elegant pharmacokinetic equations have been derived for intravenous procaine, but none have accounted for intraneural ion trapping effects of charged procaine within the dorsal root ganglia nor low CSF pseudocholinesterase activity, which predicts the effective treatment of chronic pain with oral procaine administration as described in this invention.[33] Therefore, the short distribution half-life of ˜2.5 minutes and short elimination half-life of ˜7.7 minutes of procaine does not preclude its use as an oral analgesic for chronic pain, which teaches away from what is obvious to one familiar with the prior art.

The major side effect of procaine administration is convulsions, not cardiotoxicity. The extensive protective effect of benzodiazepines on local anesthetic toxicity is well established in animal models and presumed to be present in humans.[34, 35] In an animal model, an intravenous injection of 0.3 mg/kg of diazepam aborted procaine induced convulsion from 100-150 mg/kg procaine administered intraperitoneally.[36] From these data, an approximate initial starting total dose of 10-20 mg of diazepam in divided administrations would be prophylaxis for procaine induced seizures. Therefore, with concurrent or pretreatment of a benzodiazepine, the therapeutic ratio of oral procaine for the treatment of chronic pain becomes much more favorable. Since procaine and diazepam are FDA approved medications, enablement of this invention would only require an off label clinical trial, a common investigation. If oral bioavailability in some patients is erratic, a controlled release preparation to be administered orally or subcutaneously would not be too difficult to develop.[37, 38]

Patients with intractable chronic pain defined as chronic pain that is not amenable to conventional therapies such as behavioral modification, injection, stimulation, and standard medication are potential candidates for oral procaine therapy. Prior to initiation of oral procaine therapy, chronic pain is assessed on a number scale, a blood dibucaine number is obtained to determine plasma esterase activity, procaine hypersensitivity testing or history is performed, a benzodiazepine is administered, and an EKG is interpreted.

### OTHER EMBODIMENTS OF THIS INVENTION

1. Modification of oral procaine administration to include oral double salt procaine administration,
2. Modification of oral procaine administration to include oral 2-chloroprocaine administration,
3. and modification of oral procaine administration to include a timed release procaine administration.

Oral Administration of an Anticholinesterase Inhibitor

In rare instances, adequate procaine blood levels may not be attainable secondary to increased pseudocholinesterase activity from an atypical enzyme or increased expression. Administration of an oral peripheral cholinesterase inhibitor such as neostigmine or pyridostigmine may improve the analgesic effect of oral procaine administration.[39]

### REFERENCES

1. Goldberg, J. S., PDLA a potential new potent topical analgesic: a case report. Local Reg Anesth, 2014. 7: p. 59-61.
2. Goldberg, J. S., USE OF POLYMER D-LACTIC ACID TO TREAT PAIN, US 2015/0182481 A1, 2015.
3. Goldberg, J. S., INHIBITION OF GLYCOLYSIS WITH 2-DEOXY-D-GLUCOSE AND D-LACTIC ACID DIMER, US 2020/0276216 A1, 2020.
4. Goldberg, J. S., AEROBIC GLYCOLYSIS AND HYPERMETABOLIC STATES, US 2022/0206017 A1, 2022.
5. Nguyen, P. T. and V. Yarov-Yarovoy, Towards Structure-Guided Development of Pain Therapeutics Targeting Voltage-Gated Sodium Channels. Front Pharmacol, 2022. 13: p. 842032.
6. Ma, R. S. Y., et al., Voltage gated sodium channels as therapeutic targets for chronic pain. J Pain Res, 2019. 12: p. 2709-2722.
7. Haberberger, R. V., et al., Human Dorsal Root Ganglia. Front Cell Neurosci, 2019. 13: p. 271.
8. Roos, A. and W. F. Boron, Intracellular pH. Physiol Rev, 1981. 61(2): p. 296-434.
9. Reuter, U. R. M., Oettmeier, R., Nazlikul, H., Procaine and Procaine-Base-Infusion: A Review of the Safety and Fields of Application after Twenty Years of Use. Sci Forschen 2017. 4, 1.
10. Backonja, M. and K. A. Gombar, Response of central pain syndromes to intravenous lidocaine. J Pain Symptom Manage, 1992. 7(3): p. 172-8.
11. Keats, A. S., G. L. D'Alessandro, and H. K. Beecher, Controlled study of pain relief by intravenous procaine. J Am Med Assoc, 1951. 147(18): p. 1761-3.
12. Kahl, S., et al., Procaine hydrochloride fails to relieve pain in patients with acute pancreatitis.

Digestion, 2004. 69(1): p. 5-9.
13. Zeluff, R. J., Administration of procaine intravenously; in traumatic surgery. U S Armed Forces Med J, 1950. 1(1): p. 26-9.
14. Shanbrom, E., Treatment of herpetic pain and postherpetic neuralgia with intravenous procaine.

JAMA, 1961. 176: p. 1041-3.
15. Betcher, A. M., The use of intravenous procaine in orthopaedic conditions. Bull Hosp Joint Dis, 1948. 9(1): p. 39-47.
16. Seifen, A. B., et al., Pharmacokinetics of intravenous procaine infusion in humans. Anesth Analg, 1979. 58(5): p. 382-6.
17. Klein, R. and S. B. Harris, Oral procaine for musculoskeletal pain. N Y State J Med, 1953. 53(23): p. 2832-4.
18. Kupperman, H. G. and H. Bartfeld, Oral procaine for musculoskeletal pain. N Y State J Med, 1957. 57(15): p. 2520-1.
19. Koch, R., PROCAINE DOUBLE SALT COMPLEXES, U.S. Pat. No. 5,162,344, 1992.
20. Balfour, D. C., Jr., G. K. Wharton, and H. Sky-Peck, Use of procaine hydrochloride by mouth for gastrointestinal disorders. Gastroenterology, 1952. 22(2): p. 257-62.
21. Beinhauer, L. G., Use of oral procaine in control of pruritus. AMA Arch Derm Syphilol, 1954. 69(2): p. 188-94.
22. Traut, E. F., Procaine and procaine amide hydrochloride in skeletal pain. J Am Med Assoc, 1952. 150(8): p. 785-9.
23. Ulbrich, A. P. and D. Koprince, The use of oral procaine for pruritus. J Am Osteopath Assoc, 1954. 53(12): p. 674-5.
24. Luddecke, H., Oral administration of procaine with ascorbic acid, with special reference to the therapy of pruritus. AMA Arch Derm Syphilol, 1951. 64(1): p. 9-11.
25. Schapiro, M. M. and M. Sadove, Oral procaine hydrochloride therapy in asthma. Ann Allergy, 1950. 8(1): p. 85-9.
26. Peterson, C. G., Neuropharmacology of procaine. I. Peripheral nervous actions. Anesthesiology, 1955. 16(5): p. 678-98.
27. Peterson, C. G., Neuropharmacology of procaine. II. Central nervous actions. Anesthesiology, 1955. 16(6): p. 976-93.
28. Foldes, F. F. and N. P. Mc, 2-Chloroprocaine: a new local anesthetic agent. Anesthesiology, 1952. 13(3): p. 287-96.
29. Yamada, T., et al., Localization of an isoform of carboxylesterase in rat brain differs from that in human brain. Brain Res, 1995. 674(1): p. 175-9.
30. Ruffin, V. A., et al., Intracellular pH regulation by acid-base transporters in mammalian neurons. Front Physiol, 2014. 5: p. 43.
31. Kambam, J. R., et al., Pseudocholinesterase activity in human cerebrospinal fluid. Anesth Analg, 1989. 68(4): p. 486-8.
32. Vlachova, V., et al., Procaine excites nociceptors in cultures from dorsal root ganglion of the rat. Neurosci Lett, 1999. 263(1): p. 49-52.
33. Smith, R. H., et al., Pharmacokinetic model for procaine in humans during and following intravenous infusion. J Pharm Sci, 1979. 68(8): p. 1016-22.
34. de Jong, R. H. and J. D. Bonin, Benzodiazepines protect mice from local anesthetic convulsions and deaths. Anesth Analg, 1981. 60(6): p. 385-9.
35. Sawaki, K., et al., Effects of anticonvulsants on local anaesthetic-induced neurotoxicity in rats. Pharmacol Toxicol, 2000. 86(2): p. 59-62.
36. De Jong, R. H. and J. E. Heavner, Diazepam prevents local anesthetic seizures. Anesthesiology, 1971. 34(6): p. 523-31.
37. Sokolsky-Papkov, M., et al., Prolonged local anesthetic action through slow release from poly (lactic acid co castor oil). Pharm Res, 2009. 26(1): p. 32-9.
38. Masters, D. B. and A. J. Domb, Liposphere local anesthetic timed-release for perineural site application. Pharm Res, 1998. 15(7): p. 1038-45.
39. Sunew, K. Y. and R. G. Hicks, Effects of neostigmine and pyridostigmine on duration of succinylcholine action and pseudocholinesterase activity. Anesthesiology, 1978. 49(3): p. 188-91.

Having described my invention, I claim:

1. A method of blocking a voltage gated sodium channel in a dorsal root ganglia in a human subject comprising:
measuring a dibucaine number in a blood sample of the subject;
administering an oral benzodiazepine or an equivalent, derivative, or analog thereof to the subject; and
administering an oral procaine salt or an equivalent, derivative or analog thereof to the subject.

2. The method of claim 1, wherein an oral benzodiazepine or an equivalent, derivative, or analog thereof comprises an oral dose of diazepam in a range of 0.1 mg/kg to 0.5 mg/kg.

3. The method of claim 1, wherein an oral procaine salt or an equivalent, derivative or analog thereof comprises an oral dose of procaine HCl in a range of 10 mg/kg to 100 mg/kg.

4. The method of claim 1, wherein a voltage gated sodium channel comprises a Na v 1.8 channel.

5. The method of claim 1, wherein an oral benzodiazepine or an equivalent, derivative, or analog thereof comprises an oral dose of diazepam in a range of 0.1 mg/kg to 0.5 mg/kg; wherein an oral procaine salt or an equivalent, derivative or analog thereof comprises an oral dose of procaine HCl in a range of 10 mg/kg to 100 mg/kg; and wherein a voltage gated sodium channel comprises a Naᵥ 1.8 channel.

6. A method of a chronic pain treatment in a human subject comprising:
measuring a dibucaine number in a blood sample of the subject;
administering an oral benzodiazepine or an equivalent, derivative, or analog thereof to the subject; and
administering an oral procaine salt or an equivalent, derivative or analog thereof to the subject.

7. The method of claim 6, wherein an oral benzodiazepine or an equivalent, derivative, or analog thereof comprises an oral dose of diazepam in a range of 0.1 mg/kg to 0.5 mg/kg.

8. The method of claim 6, wherein an oral procaine salt or an equivalent, derivative or analog thereof comprises an oral dose of procaine HCl in a range of 10 mg/kg to 100 mg/kg.

9. A pharmaceutical composition adapted for an oral administration for a chronic pain treatment comprising an effective amount of a procaine salt or an equivalent, derivative or analog thereof and an effective amount of a benzodiazepine equivalent, derivative or analog thereof.

10. A pharmaceutical composition as in claim 9, wherein said effective amount of a procaine salt or an equivalent, derivative or analog thereof comprises a dose of procaine HCl in a range of 10 mg/kg to 100 mg/kg.
